(19) United States
(12) Patent Application Publication
(10) Pub. No.: US 2005/0090882 A1
(43) Pub. Date: Apr. 28, 2005

(54) Hot compress underpants

(76) Inventor: Li-Chen Wei, Changhua City (TW)

Correspondence Address: TROXELL LAW OFFICE PLLC, SUITE 1404, 5205 LEESBURG PIKE, FALLS CHURCH, VA, 22041, US

(21) Appl. No.: 10/973,285

(22) Filed: Oct. 27, 2004

(30) Foreign Application Priority Data
Oct. 28, 2003 (TW) 092219124

(51) Int. Cl. A61F 7/00

(52) U.S. Cl. 607/108; 607/114

(57) ABSTRACT

A pair of hot compress underpants with a hot compress piece, comprising an underpants body, the main body is provided on a front portion thereof with a hot compress pocket for receiving a hot compress piece. The hot compress pocket is connected with the main body to form a space with the front portion of the main body, thereby the hot compress piece can be received in from an opening of the hot compress pocket. The position of the hot compress pocket is located exactly in the lower abdomen of a wearer, thus the effect of easing painful menstruation of a wearer can be obtained.

### BACKGROUND OF THE INVENTION

1. Field of the Invention

The invention is related to a pair of hot compress underpants, especially with a hot compress piece for easing menstrual cramps by the heat transpiring from the hot compress function.

2. Description of the Prior Art

Generally, most women feel slight ache on their waists and unwell on their lower abdomens during their menstruation. These kinds of symptoms will not make any influences on their day-to-day life and work. However, some women will have severe symptoms such as lower-abdomen aches, waist aches, disgusting and vomiting etc. that will affect their day-to-day life and work before or after menstruations. Those severe symptoms are called dysmenorrhea or menstruation cramps. Most of the cramps occurred in the lower abdomens, however, some of them will be spread to the waist, the anus and the perineum. Usually women have menstruation cramps will have unbearable throes. In serious cases, those symptoms may be accompanied by phenomenon of disgusting, vomiting, pale face, breaking out in cold sweat, headache, fidgeting and agitation etc.

The common way of easing cramps is to keep the painful area warm. Compressing the painful area lightly with a hot towel or a hot water bag lightly to help speed up the blood circulation, the pain will be alleviated then. However, both of the hot towel and the hot water bag need hot water that may result in inconveniences. Besides, the effective duration of the hot towel is short, and the hot towel will wet the clothes.

There have been many known hot compress devices in the markets. Some may generate heat by making contact with the air, and some by the principle of infrared heating. The rear of such a hot compress device is provided with an adhesive surface for sticking the portable hot compress piece at a painful area of the lower abdomen on the inner layer of a pair of underpants. The time span of such a hot compress piece can persist for about 5 to 8 hours, which is quite effective in alleviating the state of discomfort. Therefore, the pain and discomfort entailed during monthly periods can be made more bearable through this means.

However, due to the fact that such hot compress piece is intended to be stuck on the inner layer of a pair of underpants, when a woman in her monthly periods goes to lavatory or needs to change her sanitary pad, she may cause the hot compress piece to be displaced or loosen its adhesion while wearing or taking off the underpants. This situation is quite bothersome. Therefore, a means for reliable attachment is needed to ensure that the hot compress piece would be firmly mounted in the inner layer of the pair of underpants. Furthermore, some women are not accustomed to have their skins directly in contact with hot compress pieces, and some women's skins may develop allergic reactions such as irritation etc. The mode in the present invention can help solve the above-mentioned problems.

### SUMMARY OF THE INVENTION

In view of the above defects, the inventor devised a pair of hot compress underpants, able to receive therein a hot compress piece that can be reliably and firmly mounted on the inner layer of the pair of underpants without bothersome dropping, which permits women to wear during their monthly periods in a pleased and calm mood.

The primary objective of the present invention is to provide a pair of hot compress underpants able to receive, therein, a hot compress piece. The pair of hot compress underpants is composed of a main body of the underpants, which is provided on its front portion, thereon, with a hot compress pocket for receiving a hot compress piece. The hot compress pocket is sewed or hot-melted with the main body and forms a space with the front portion of the main body, thereby the hot compress piece can be received in from an opening of the hot compress pocket. The position of the hot compress pocket is located at the lower abdomen of a wearer such that the hot compress piece can entail its maximum effect at easing painful menstruation.

Furthermore, according to another type, the present invention provides a pair of hot compress underpants having, therein, a hot compress piece. The hot compress underpants includes a main body of the underpants, and is characterized by that, the front portion of the main body of the underpants is connected with a hot compress piece for hot compressing when a wearer wears the underpants. The position of the hot compress piece is in the area of the lower abdomen of a wearer, thereby the effect of easing painful menstruation can be obtained. The connecting mode of hot compress piece with the main body of the underpants can just be sewing or hot-melting connecting. The main body of the underpants can also be a disposable type, in order that a woman can wear the disposable underpants outdoors or in traveling during her monthly periods. This is a convenient option.

The present invention will be apparent upon reading the detailed description of the preferred embodiments thereof in reference to the accompanied drawings.

### BRIEF DESCRIPTION OF THE DRAWINGS

FIG. 1 is a partial sectional schematic front view of an embodiment of a pair of hot compress underpants of the present invention;

FIG. 2 is a sectional side view taken from a sectional line A-A of FIG. 1;

FIG. 3 is a partial sectional schematic front view of another embodiment of hot compress underpants of the present invention;

FIG. 4 is a sectional side view taken from a sectional line B-B of FIG. 3.

### DETAILED DESCRIPTION OF THE PREFERRED EMBODIMENT

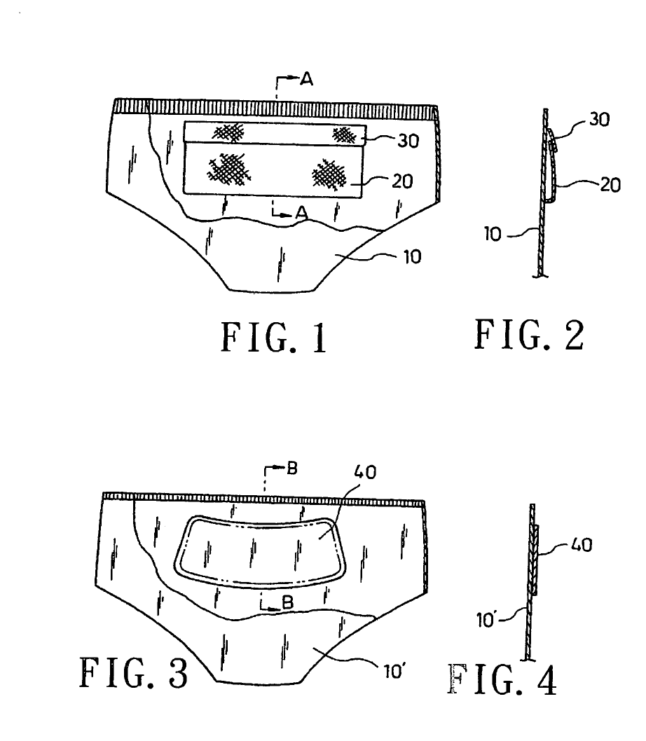

Referring to FIGS. 1 and 2, a pair of hot compress underpants provided with a hot compress piece of the present invention comprises a main body 10 of the underpants, the main body 10 is provided on the front portion thereon with a hot compress pocket 20 for receiving a hot compress piece (not shown). The hot compress pocket 20 is connected with the main body 10 by way of sewing or hot-melting connection, and is in a rectangular shape with an upward opening; it forms a space with the front portion of the main body 10, thereby the hot compress piece can be received in from the opening of the hot compress pocket 20. Material of the hot compress pocket 20 shall be light, thin and ventilative, so that heat of the hot compress piece therein can be easily transmitted to the skin of a wearer. Moreover, the position of the hot compress pocket 20 can make the position of the hot compress piece be exactly in the area of the lower abdomen of a wearer, thus accruing the effect of alleviating painful menstruation. The hot compress pocket 20 is in a rectangular shape with its size and shape adequate to receive a normal hot compress piece. However, the hot compress pocket 20 as shown in FIG. 1 is only for the purpose of illustration, the hot compress pocket s of the present invention are not limited to the shape only. Besides, the hot compress pocket 20 is provided on the upper edge thereof with a pending flap 30. When a hot compress piece loses its function and the wearer needs to change it, the wearer needs only to lift the flap 30 and to draw out the original hot compress piece, then to place a new hot compress piece into the hot compress pocket 20. By providing the flap 30 the hot compress piece can be reliably and firmly mounted in the hot compress pocket 20.

Since the pair of hot compress underpants provided with a hot compress piece of the present invention has a hot compress pocket, the hot compress pocket can reliably and firmly receive the hot compress piece at a suitable position in the main body of the underpants without worrying dropping or losing adhesion. And with the setting of the hot compress pocket, the wearer's skin does not directly contact with the hot compress piece, thus may induce an irritating allergic phenomenon. While heat of the hot compress piece can still be transmitted to the area of the lower abdomen of the wearer through the hot compress pocket 20 made of light, thin and ventilative material. Moreover, the material of the hot compress pocket 20 is so light and thin that the wearer does not feel discomfort even if wearing the hot compress underpants not in her monthly period. The hot compress underpants of the present invention provides applicability both for the normal time and the monthly periods of a wearer.

FIGS. 3 and 4 illustrate another embodiment of the present invention, a difference of this embodiment from the former embodiment resides in that, a hot compress piece 40 of this embodiment is directly sewn or stuck by hot melting connecting to the front portion of a main body 10′ of a pair of hot compress underpants without an extra hot compress pocket. This hot compress underpants is disposable, it can be discarded after using. Such an expediential design allows a woman to wear the disposable underpants outdoors or in traveling during her monthly periods, it is really convenient for use.

There have been two embodiments illustrated in the present invention. However, the present invention is not limited to them. For example, the shapes and materials can be various, the directions of opening of the hot compress pocket s can also be changed from being upward to being sideward; and the flaps are not definitely required, they can be substituted by contractible openings. It will be apparent to those skilled in this art that various equivalent modifications or changes can be made to the elements of the present invention; accordingly, all such modifications and changes without departing from the spirit and scope of this invention also fall within the scope of the appended claims and are intended to form part of this invention.

1. A pair of hot compress underpants comprising a main body and a hot compress pocket which is designed for the hot compress piece to be put in front portion of the said main body; the said pocket being connected with the main body and forming a space on the front portion of the said main body; and the said hot compress piece being allowed to be put in from the opening of the said hot compress pocket.

2. The hot compress underpants as claimed in claim 1, illustrates that the hot compress pocket is located in an area of the lower abdomen of a wearer.

3. The hot compress underpants as claimed in claim 1, illustrates that the hot compress pocket is connected with main body of the underpants by way of sewing.

4. The hot compress underpants as claimed in claim 1, illustrates that the hot compress pocket is connected with a main body of a underpants by way of hot-melting.

5. The hot compress underpants as claimed in claim 1, illustrates that the hot compress pocket has an upward opening.

6. The hot compress underpants as claimed in claim 5, illustrates that the hot compress pocket is provided at an opening with a pending flap for receiving a hot compress piece reliably and firmly in a hot compress pocket.

7. The hot compress underpants as claimed in claim 1, illustrates that the hot compress pocket has an upward opening, the opening is a contractible to prevent the hot compress piece from dropping out of the hot compress pocket.

8. The hot compress underpants as claimed in claim 1, illustrates that the hot compress pocket has an opening on one of its lateral sides.

9. A pair of hot compress underpants provided with a hot compress piece, a pair of underpants comprises a main body, a main body is provided on a front portion thereof with a hot compress piece for hot compressing on a wearer when wearing the underpants.

10. The hot compress underpants as claimed in claim 9, illustrates that the position of the hot compress piece is in an area of the lower abdomen of a wearer.

11. The hot compress underpants as claimed in claim 9, illustrates that the main body of the underpants is of a disposable type.

12. The hot compress underpants as claimed in claim 9, illustrates that the hot compress piece is connected with the main body of the underpants by way of sewing.

13. The hot compress underpants as claimed in claim 10, illustrates that the hot compress piece is connected with the main body of the underpants by way of sewing.

14. The hot compress underpants as claimed in claim 11, illustrates that the hot compress piece is connected with the main body of the underpants by way of sewing.

15. The hot compress underpants as claimed in claim 9, illustrates that the hot compress piece is connected with the main body of the underpants by way of hot-melting.

16. The hot compress underpants as claimed in claim 10, illustrates that the hot compress piece is connected with the main body of the underpants by way of hot-melting.

17. The hot compress underpants as claimed in claim 11, illustrates that the hot compress piece is connected with the main body of the underpants by way of hot-melting.
